(19) United States
(12) Patent Application Publication
(10) Pub. No.: US 2023/0052467 A1
(43) Pub. Date: Feb. 16, 2023

(54) METHODS OF PRODUCING LACTIC ACID FROM UNMODIFIED STARCH

(71) Applicant: BluCon Biotech GmbH, Koln (DE)

(72) Inventors: Tatiana Svetlichnaya, Koln (DE); Vitaly Svetlichny, Koln (DE); Marco Kramer, Pulheim (DE)

(21) Appl. No.: 17/791,752

(22) PCT Filed: Jan. 10, 2020
(86) PCT No.: PCT/EP2020/050528

§ 371 Date: Jul. 8, 2022

(51) Int. Cl. C12P 7/56; C12N 1/20

(52) U.S. Cl. CPC C12P 7/56; C12N 1/205; C12R 2001/01

(57) ABSTRACT

The technology provided herein relates to novel methods for producing lactic acid (L-lactic acid, D-lactic acid and D/L-Lactic acid) from starch containing material with extreme thermophilic bacterial cells belonging to the genus Caldicellulosiruptor, mutants thereof, isolated strains, microbial cultures, and microbial compositions. The novel methods are in particular suitable for the production of lactic acid from any carbon source, not limited to but especially useful for unmodified starch and/or starch-containing material.

### FIELD OF THE DISCLOSURE

The present disclosure pertains to novel methods for producing lactic acid (L-lactic acid, D-lactic acid and D/L-Lactic acid) from starch containing material with extreme thermophilic bacterial cells belonging to the genus Caldicellulosiruptor, mutants thereof, isolated strains, microbial cultures, and microbial compositions. The novel methods are in particular suitable for the production of lactic acid from any carbon source, not limited to but especially useful for unmodified starch and/or starch-containing material.

### BACKGROUND

Starch is a readily available renewable material being used for food and industrial applications, including syrup production, bioethanol and biochemicals production and paper production. The major sources of starch worldwide are cereals such as corn, wheat and rice, and roots such as potatoes and cassava. Starch is generally found in the leaves, seeds, roots and fibers of plants where it serves as energy reserve.

Starch is a large molecular weight polysaccharide composed of glucose molecules joined by glycosidic bonds. It consists of two structurally different molecules: amylose that makes up 20-30% of the starch, and amylopectine that makes up 70-80%. The relative contribution of each molecule is dependent on the plant source. Amylose is a linear chain of glucose units joined by α-1,4 bonds with a degree of polymerization up to 6,000, while amylopectin is a highly branched chain of glucose units with a degree of polymerization up to 2 million. The branching in amylopectin occurs every 24 to 30 glucose units using α-1,6 bonds. In general, amylose is less susceptible to degradation than amylopectine, because of the unbranched glucose chains that more readily crystallize and the lower number of chain end points onto which enzymes can attach.

The hydrolysis of starch into oligosaccharides and glucose can be achieved using enzymes, acids, or a combination of the two. Nevertheless, the enzymatic hydrolysis has so far been the most preferable method. The hydrolysis generally consists of three consecutive steps, which are referred to as gelatinization, liquefication, and saccharification. Basically, (i) gelatinization involves the disintegration of the starch granules in water at high temperatures, (ii) liquefication encompasses the partial hydrolysis of the starch by the enzyme α-amylase forming shorter-chain oligosaccharides, and (iii) saccharification involves the full hydrolysis of the oligosaccharides by the enzyme glucoamylase forming mainly glucose.

During gelatinization, the intermolecular bonds in starch are broken in the presence of water at temperatures of 90 to 165 degrees centigrade, allowing the hydrogen bonding sites to engage more water. As a consequence, the starch granules swell and burst, the semi-crystalline structure is partly lost, and smaller amylose molecules start leaching out of the granule. Hence, gelatinization irreversible dissolves the starch granule in water and increases the availability of starch for the subsequent hydrolysis by amylases.

The gelatinization process thus converts native starches into modified starches. In literature, native starches, also designated raw starches, are defined as unmodified and unprocessed starches, whereas modified starches are defined as starch products obtained by physical, enzymatic or chemical processes, which lead to changes in physicochemical properties such as moisture content, amylose content, swelling and viscosity (Karmakar et al. 2014).

After gelatinization, the starch molecules are broken down into oligosaccharides and glucose by the action of amylases (in particular, α-amylase and glucoamylase). Alpha-amylase is the first enzyme used in the hydrolysis process acting randomly along the starch chain breaking down the α-1,4 glycosidic bonds to produce oligosaccharides, maltose and glucose. Glucoamylase, the second enzyme in the hydrolysis process, hydrolyses both α-1,4 and α-1,6 bonds from the non-reducing end of oligosaccharides and maltose to produce glucose.

Numerous bacteria and fungi exist that naturally produce α-amylase and glucoamylase. With regard to α-amylase, the enzyme has been found mostly in bacterial cultures of Bacillus spp. like Bacillus amyloliquefaciens, Bacillus licheniformis, Bacillus subtilis and Bacillus megaterium, and fungal cultures of Aspergillus spp. like Aspergillus oryzae and Aspergillus niger. With regard to glucoamylase, the enzyme is produced by a few Bacillus spp. and by the fungal species Aspergillus niger, Aspergillus oryzae, Aspergillus saitai and Aspergillus awamori.

As mentioned before, starch is an easily accessible carbon source to be used in industrial biotechnological applications. In this context, ‘starch’ should be understood in its broadest sense, comprising not only starch containing crops and agricultural residues, but also different types of starch containing waste streams from industry and households.

By far the most important application of starch (in terms of volume) is the production of bioethanol from corn using the yeast Saccharomyces cerevisiae. The yeast cells are traditionally added to the starch solution at the start of the saccharification process with glucoamylase, and progressively converts the released glucose molecules into ethanol and carbon dioxide. This process, which is referred to as simultaneous saccharification and fermentation (SSF), is performed at temperatures of 30-35 degrees centigrade and takes approximately 72 hours, resulting in an ethanol concentration of 16-18% (v/v). Afterwards, the yeast cells are separated from the fermentation broth by centrifugation and the ethanol is recovered by distillation.

SSF has several advantages as compared to a process with sequential hydrolysis and fermentation. Some of the advantages are the use of only a single vessel for hydrolysis and fermentation, thus reducing the investment costs and residence times. Another advantage is the reduction of end-product inhibition of the enzymatic hydrolysis, thus improving the overall process performance. The main disadvantage is the challenge to find favorable operation conditions for both hydrolysis and fermentation. In most cases, the applied enzymes and microorganisms have different optimal conditions (such as pH and temperature) for maximal performance, and thus a less-optimal midway has to be found and applied.

Apart from the price of the substrate, the cost of the production process is an important factor in the commercial viability of industrial biotechnological applications, such as the above-mentioned production of bioethanol or the production of other biochemicals such as lactic acid and n-butanol. As each step in the production process adds to the cost, reducing the number of steps would thus positively impact the economics of the overall process.

As a follow-up of SSF, consolidated bioprocessing (CBP) is a process applying organisms that combine the production of enzymes needed for complete substrate hydrolysis and the fermentation of the released sugars in a single step. Although CBP has not yet been implemented on a commercial scale (mainly because no microorganism is currently available that expresses all necessary enzymes for hydrolysis and shows a high fermentation performance), the process would simplify operational processes, and thus reduce maintenance and production costs.

Lactic acid is an interesting biochemical to be produced by CBP on a commercial scale. Lactic acid is used throughout the world in manufacturing of food, chemicals, and pharmaceutical products. Recently, there is a lot of interest in biodegradable poly-lactic acid, which is an alternative to petrochemically derived plastic (Drumright et al. 2000). Chiral pure lactic acid is produced commercially by microbial fermentation of the carbohydrates glucose, sucrose, lactose, and starch/maltose derived from feedstocks such as beet sugar, molasses, whey, and barley malt (Narayanan et al. 2004). The choice of feedstock depends on its price, availability, and on the respective costs of lactic acid recovery and purification (Datta et al. 1995; Vaidya et al. 2005).

In a study of Panda and Ray (2008), lactic acid production was carried out by the Lactobacillus plantarum strain MTCC 1407 in semi-solid fermentation using sweet potato (Ipomoea batatus L.) flour. Notably, sweet potato flour is a readily available and cheap source of carbon and other nutrients. It was shown that the amylolytic strain is able to convert the raw starch present in the sweet potato flour to lactic acid in a single step. The organism produced 23.86 g of lactic acid from 55 g of starch (43.4%) present in 100 g of sweet potato flour, showing 56% conversion after 120 hours of incubation. Other Lactobacillus species and strains reported to produce lactic acid from starch containing materials are Lactobacillus plantarum ATCC 21028 (Fu and Mathews 1999), Lactobacillus casei (John et al. 2007), and Lactobacillus amylophilus GV6 (Reddy et al. 2008, Naveena et al. 2004). Naveena et al. (2005) reported that Lactobacillus amylophilus GV6 produces 36 g of lactic acid from 54.4 g of starch present in 100 g of wheat bran with a yield of 77.6%.

In a study of Narita et al. (2004), it was found that Streptococcus bovis could directly produce lactic acid from starch, resulting in a lactic acid concentration of 14.2 g/L. In another study, a starch degrading strain of Lactobacillus casei was constructed by genetically displaying α-amylase from Streptococcus bovis with a FLAG peptide tag (AmyAF) (Narita et al. 2006, Reddy et al. 2008). In the process of fermentation using AmyAF displaying Lactobacillus. casei cells, 50 g/L of soluble starch was reduced to 13.7 g/L starch, and 21.8 g/L of lactic acid was produced within 24 hours.

In addition to the above described members of the family Lactobacillales, Wang et al. (2019) described a one-step production process for L-lactic acid using Bacillus coagulans, a species belonging to the family Bacillaceae. In particular, about 50 g/l lactic acid was produced from 72 g/l soluble starch at 52 degrees centigrade. Since Bacillus coagulans is tolerant to high temperatures (58 degrees centigrade), a CBP system with an open fermentation operation could be envisaged.

In addition to the above described process with Bacillus coagulans a direct fermentation process of raw starch and modified starch; i.e. potato starch and potato residues to lactic acid under non-sterile conditions was carried out by Geobacillus stearothermophilus (Smerilli et al., 2015). In this process, production of lactic acid was carried out at 60 degrees centigrade.

In fact, several amylolytic lactic acid bacteria (Lactobacillales) are known to produce lactic acid from starch containing materials. Most of these bacteria belong to the genera Lactobacillus, Lactococcus, Streptoccocus, Pediococcus, Carnobacterium and Weissella (Bhanwar and Ganguli 2014). A few examples of the fermentation processes are given below.

Biohydrogen production from modified starch, which was heat treated and hydrolyzed potato steam peels was carried out with Caldicellulosiruptor saccharolyticus at 72 degrees centigrade (Mars et al., 2010).

Such systems would largely reduce energy consumption as no sterilization is needed and less cooling is required during fermentation.

In all of the above studies, different renewable starch containing materials were used for production of lactic acid. In this regard, using cheap inedible starch containing materials are an interesting alternative to edible materials such as corn, rice and potatoes. Native unprocessed starch containing material will be potential cheaper than modified processed starch. Such materials will not only reduce the costs of the production process, but also circumvent the competition for food. Examples of inedible starch containing materials are waste streams from the bread and dough industry, the potato processing industry, and the grain milling industry.

Therefore, the availability of novel methods for converting unmodified starch-containing biomass material to lactic acid would be highly advantageous.

### SUMMARY OF THE DISCLOSURE

The present invention relates to novel fermentation processes using thermophilic microorganisms of the genus Caldicellulosiruptor and compositions useful for converting native (unmodified) starch-containing materials to lactic acid, which can either be the two enantiomers L-lactic acid or D-lactic acid or the racemic mixture of D- and L-lactic acid.

In a first aspect, the present disclosure pertains to a fermentation process for the production of lactic acid comprising the steps of contacting unmodified starch and/or unmodified starch-containing material with a microbial culture comprising a microorganism of the genus Caldicellulosiruptor for a period of time at an initial temperature and an initial pH, thereby producing an amount of a lactic acid, wherein the unmodified starch and/or the unmodified starch-containing material is converted in a single step process as part of a consolidated bioprocessing (CBP) system.

In particular, in the process according to the present disclosure the lactic acid is separated during and/or after the conversion.

Starch containing material can be distinguished between native starch materials, also designated raw starch materials, are unmodified and unprocessed starch material, whereas modified starch materials are starch materials obtained by physical, enzymatic or chemical processes, which lead to changes of physicochemical properties like moisture content, amylose content, swelling and viscosity and other parameters (Karmakar et al., 2014).

Native and modified starch-containing materials (e.g., biomass materials or biomass-derived materials, such as native starchy materials (native starch), or biomass materials that include significant amounts of low molecular weight sugars, which are degradation products of native or modified starch (e.g., monosaccharides, disaccharides, or trisaccharides), can be processed to change their structure, and products can be made from the structurally changed materials.

For example, many of the methods described herein can provide starch-containing materials that have a lower molecular weight and/or crystallinity relative to a native material. Many of the methods provide materials that can be more readily utilized by a variety of microorganisms to produce useful products, such as organic acids (e.g., lactic acid), hydrogen, alcohols (e.g., ethanol or butanol), hydrocarbons, co-products (e.g., proteins) or mixtures of any of these.

In a first aspect, embodiments of the disclosure provide isolated extreme thermophilic bacterial cells belonging to the genus Caldicellulosiruptor, in particular capable of producing high levels of lactic acid from starch containing materials, e.g. biomass.

In one aspect, embodiments of this disclosure relate to all microorganisms of the genus Caldicellulosiruptor, species and strains of the genus Caldicellulosiruptor. These include Caldicellulosiruptor acetigenus, Caldicellulosiruptor bescii, Caldicellulosiruptor changbaiensis, Caldicellulosiruptor danielii, Caldicellulosiruptor sp. strain F32, Caldicellulosiruptor hydrothermalis, Caldicellulosiruptor kristjanssonii, Caldicellulosiruptor kronotskyensis, Caldicellulosiruptor lactoaceticus, Caldicellulosiruptor morganii, Caldicellulosiruptor naganoensis, Caldicellulosiruptor obsidiansis, Caldicellulosiruptor owensensis and Caldicellulosiruptor saccharolyticus, Caldicellulosiruptor sp. str. DIB 041C DSM 25771, Caldicellulosiruptor sp. str. DIB 004C DSM 25177, Caldicellulosiruptor sp. str. DIB 101C DSM 25178, Caldicellulosiruptor sp. str. DIB 103C DSM 25773, Caldicellulosiruptor sp. str. DIB 107C DSM 25775, Caldicellulosiruptor sp. str. DIB 087C DSM 25772 and Caldicellulosiruptor sp. str. DIB 104C DSM 25774, Caldicellulosiruptor sp. BluCon006 DSM 33095, Caldicellulosiruptor sp. BluCon014 DSM 33096, Caldicellulosiruptor sp. BluCon016 DSM 33097 and Caldicellulosiruptor sp. BluConL60 DSM

In still another aspect the present invention relates to a cell of the genus Caldicellulosiruptor according to any of the preceding aspects.

In a further aspect, embodiments of this disclosure relate to microorganism of the strains Caldicellulosiruptor sp. str. DIB 041C DSM 25771, Caldicellulosiruptor sp. str. DIB 004C DSM 25177, Caldicellulosiruptor sp. str. DIB 101C DSM 25178, Caldicellulosiruptor sp. str. DIB 103C DSM 25773, Caldicellulosiruptor sp. str. DIB 107C DSM 25775, Caldicellulosiruptor sp. str. DIB 087C DSM 25772 and Caldicellulosiruptor sp. str. DIB 104C DSM 25774, Caldicellulosiruptor sp. BluCon006 DSM 33095, Caldicellulosiruptor sp. BluCon014 DSM 33096, Caldicellulosiruptor sp. BluCon016 DSM 33097 and Caldicellulosiruptor sp. BluConL60 DSM 33252 microorganism derived therefrom, progenies or mutants thereof, wherein the mutants thereof retaining the properties of the genus Caldicellulosiruptor.

In still another aspect, embodiments of this disclosure relate to methods for converting native or modified starch-containing material like native or modified starch or native or modified starch containing biomass to a carbon-based chemical, in particular lactic acid and/or a salt or an ester thereof, comprising the step of contacting the native or modified starch containing biomass material with a microbial culture for a period of time at an initial temperature and an initial pH, thereby producing an amount of a carbon-based products, in particular lactic acid and/or a salt or an ester thereof; wherein the microbial culture comprises an extremely thermophilic microorganism of the genus Caldicellulosiruptor or a species or strain or culture of the genus Caldicellulosiruptor, in particular all microorganisms of the genus Caldicellulosiruptor, microorganisms derived from either of these strains and cultures or mutants or homologues thereof, in particular mutants thereof retaining the properties.

In still another aspect, embodiments of this disclosure relate to methods of making lactic acid from a carbon-based biomass like native or modified starch and native or modified starchy based biomass material, wherein the method comprises combining a microbial culture and the biomass in a medium; and fermenting the biomass under conditions and for a time sufficient to produce lactic acid, a salt or an ester thereof, in a single step process as part of a consolidated bioprocessing (CBP) system, with a cell, strain, microbial culture and/or a microorganism according to the present disclosure under suitable conditions, in particular using mutants thereof retaining the properties.

In still another aspect, embodiments of this disclosure relate to methods of making lactic acid from native or modified starch or native or modified starchy biomass material, wherein the method comprises combining a microbial culture and the biomass in a medium; and fermenting the biomass under conditions and for a time sufficient to produce lactic acid, a salt or an ester of the latter, in a single step process as part of a consolidated bioprocessing (CBP) system, with a cell, strain, microbial culture and/or a microorganism or mutants thereof retaining the properties according to the present disclosure under suitable conditions.

In still another aspect, embodiments of this disclosure relate to methods of making lactic acid from native or modified starchy biomass material, wherein the method comprises combining a microbial culture and the biomass in a medium; and fermenting the biomass under conditions and for a time sufficient to produce lactic acid, a salt or an ester of the latter, in a single step process as part of a consolidated bioprocessing (CBP) system, with a cell, strain, microbial culture and/or a microorganism according to the present disclosure under suitable conditions.

In an advantageous embodiment, the fermentation process of the present disclosure for the production of lactic acid is a consolidated bioprocessing (CBP) process. In particular, in advantageous embodiments no additional enzymes for liquefaction and/or for hydrolyzing the starch material like alpha amylases and/or amyloglucosidases are added before and/or during the fermentation process of the present disclosure.

Further, embodiments of this disclosure relate to compositions for converting carbon-based biomass material like native or modified starch or native or modified starchy biomass or a microbial culture comprising a cell, strain or microorganism according to the present disclosure.

Further, embodiments of this disclosure relate to the use of a cell, strain, microorganism and/or a microbial culture according to the present disclosure for the production of lactic acid, a salt or an ester thereof.

In still another aspect, embodiments of this disclosure relate to a lactic acid production procedure, characterized in that it includes the following steps:
a) converting unmodified starch and/or unmodified starch-containing material to lactic acid in a single step process as part of a consolidated bioprocessing (CBP) system in a bioreactor by a microorganism of the genus Caldicellulosiruptor,
b) separation of lactic acid from the fermentation medium
c) purification of lactic acid.

Before the disclosure is described in detail, it is to be understood that this disclosure is not limited to the particular component parts of the devices described or process steps of the methods described and methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

To provide a comprehensive disclosure without unduly lengthening the specification, the applicant hereby incorporates by reference each of the patents and patent applications cited herein.

### BRIEF DESCRIPTION OF THE DRAWINGS

FIG. 1: Graph showing the lactic acid concentrations from unmodified pure potato starch by BluConL60 at different cultivation times.

FIG. 2: Graph showing the lactic acid concentrations from unmodified pea starch by BluConL60 at different cultivation times.

FIG. 3: Graph showing the lactic acid concentrations from unmodified wheat starch by BluConL60 at different cultivation times.

FIG. 4: Graph showing the lactic acid concentrations from unmodified tapioca (cassava) starch by BluConL60 at different cultivation times.

FIG. 5: Graph showing the lactic acid concentrations from unmodified corn (maize) starch by BluConL60 at different cultivation times.

### DETAILED DESCRIPTION OF THIS DISCLOSURE

The present disclosure relates to methods, microorganisms, and compositions useful for processing native or modified starch or native or modified starchy containing biomass. The disclosure relates, in certain aspects, to microorganisms which are able to convert native or modified starchy biomass such as, for example ground crops like peas, rice, wheat, cassava and potato, to soluble products that can be used by the same or by another microorganism to produce an economically desirable product such as, for example, a carbon-based chemical, in particular lactic acid and/or a salt thereof.

An advantage property of members of the genus Caldicellulosiruptor is the high temperature tolerance, which is higher than 70 degrees for fermentative lactic acid production, which is a higher temperature tolerance compare to the members of the family of the Lactobacillales and members of the family of the Bacillaceae. All members of the genus Caldicellulosiruptor could be used for the conversion processes.

The application of this technology has the potential to render production of carbon-based chemicals more economically feasible and to allow a broader range of microorganisms to utilize starchy biomass. The use of native and modified starch containing biomass as sources of carbon-based chemicals like lactic acid is currently limited by typically requiring preprocessing, e.g. addition of amylases and glucoamylases of the starch containing material. Such preprocessing methods can be expensive. Thus, methods that reduce dependence on preprocessing of native and modified starch containing materials may have a dramatic impact on the economics of the use of native containing biomass for carbon-based chemicals production.

Usually starch-containing material like starch-containing biomass is contaminated with lignocellulose containing material, e.g. in wheat bran.

Since Caldicellulosiruptor species are not only able to utilize starch, but also able to convert lignocellulosic biomass into fermentation products a fermentative process of for using this heterogenic biological substrate material is advantages.

Native starches, also designated raw starches, are unmodified and unprocessed starches, whereas modified starches are starch products obtained by physical, enzymatic or chemical processes, which lead to changes of physicochemical properties like moisture content, amylose content, swelling and viscosity (Karmakar et al., 2014). As a result, modified starches require higher costs of energy and time for starch modification processes compared to using native starch making fermentation processes using native starches attractive.

The present inventors have found microorganisms of the genus Caldicellulosiruptor which have a variety of advantageous properties for their use in the conversion of native (unmodified) starch containing biomass/material to carbon-based chemicals, preferably to lactic acid and/or a salt thereof, preferably in a single step process as part of a consolidated bioprocessing (CBP) system.

In particular, these microorganisms are extremely thermophilic and show broad substrate specificities and high natural production of lactic acid. Moreover, lactic acid fermentation at high temperatures, for example over 70 degrees centigrade has many advantages over mesophilic fermentation. One advantage of thermophilic fermentation is the minimization of the problem of contamination in batch cultures, fed-batch cultures or continuous cultures, since only a few microorganisms are able to grow at such high temperatures in un-detoxified starch biomass material.

Another aspects of fermentations at high temperatures is that viscosity of the culture is dramatically reduced decreasing the required electric energy input for stirring. Additionally, energy for cooling of the process is not necessary.

It is also an advantage that the cells, strains and microorganisms according to the present disclosure grow on pre-treated as well as on untreated lignocellulosic biomass material. Furthermore, the cells, strains and microorganisms according to the present disclosure produces high lactic acid concentrations and low acetic acid and ethanol concentrations in fermentation processes by converting starch containing biomass/material.

In particular, the strain is Caldicellulosiruptor sp. BluConL60 produces high lactic acid concentrations after a cultivation time of 36 to 100 hours or more.

In the present context, the term “starch-containing biomass” or “starch-containing material” includes in particular starch-containing plant material, including tubers, roots, whole grain; and any combination thereof. The starch-containing biomass/material may be obtained from cereals. Suitable starch-containing biomass/material includes corn (maize), wheat, barley, cassava, sorghum, rye, potato, peas or any combination thereof. Pea is the preferred feedstock. The starch-containing material may also consist of or comprise, e.g., a side stream from starch processing, e.g., C6 carbohydrate containing process streams that may not be suited for production of syrups. Whole stillage typically contains about 10-15 wt-% dry solids. Whole stillage components include fiber, hull, germ, oil and protein components from the starch-containing feedstock as well as non-fermented starch.

In an advantageous embodiment, the unmodified starch is derived from peas and the starch-containing material are peas, parts of peas and/or pea-containing material.

The pretreatment method most often used for starchy biomass processing is steam pretreatment, a cooking process. This process used in dry milling, grain ethanol production, and other industrial starch processing applications comprises heating of the starch containing material by steam injection by a jet cocker to a temperature of 105 degrees centigrade with or without subsequent sudden release of pressure.

In the primary liquefaction stage, slurry is then pumped through a pressurized jet cooker at 105 degrees centigrade and held for 5 minutes. The mixture is then cooled by an atmospheric or vacuum flash condenser. After the flash condensation cooling, the mixture is held for 1-2 hours at 80 to 90 degrees centigrade to allow the enzymes like thermophilic amylases time to work.

It has been found that the microorganisms according to the present disclosure can grow efficiently on various types of pretreated and untreated starchy biomass (e.g. cassava, sweet potato, yam, aroids, sugar beet, peas).

“Amylolytic enzymes” are a group of starch-degrading enzymes that include the industrial important amylases, and a number of enzymes with potential applications, such as pullulanase, α-glucosidase, and cyclodextrin glycosyltransferase. Amylases have found major applications in the starch sweetener industry. α-Amylase is used in the liquefaction step producing soluble dextrins, while glucoamylase further hydrolyzes the dextrins to glucose in the saccharification step. β-Amylase is used in the production of high-maltose syrups. These enzymes also play an important role in the brewing industry, in distilleries and in the baking process.

As used herein “efficient” growth refers to growth in which cells may be cultivated to a specified density within a specified time.

As used herein “unmodified starch” or “unmodified starch-containing material” or “native starch” refers to unmodified and unprocessed starch material which are not obtained by physical including heat treatment, enzymatic or chemical processes, which lead to changes of physicochemical properties like moisture content, amylose content, swelling and viscosity and other parameters (Karmakar et al., 2014). In particular, the unmodified starch or unmodified starch-containing biomass/material is not heat treated before conversion with a method/process/procedure according to the present disclosure.

The microorganisms according to the present disclosure can grow efficiently on native and modified starch. The main product when grown on untreated biomass substrates was lactic acid.

The microorganisms according to the present disclosure also can grow efficiently on spent biomass—insoluble material that remains after a culture has grown to late stationary phase (e.g., greater than 10⁸ cells/mL) on untreated biomass.

Furthermore, the microorganisms according to the present disclosure grew efficiently on both the soluble and insoluble materials obtained after heat-treating the biomass.

The microorganisms according to the invention are anaerobic thermophile bacteria, and they are capable of growing at high temperatures even at or above 70 degrees centigrade. The fact that the strains are capable of operating at this high temperature is of high importance in the conversion of starch containing biomass into fermentation products. The conversion rate of carbohydrates into e.g. lactic acid is much faster when conducted at high temperatures. For example, the volumetric ethanol productivity of a thermophilic Bacillus is up to ten-fold higher than a conventional yeast fermentation process which operates at 30 degrees centigrade Consequently, a smaller production plant is required for a given plant capacity, thereby reducing plant construction costs. As also mentioned previously, the high temperature reduces the risk of contamination from other microorganisms, resulting in less downtime and increased plant productivity. The high operation temperature may also facilitate the subsequent recovery of the resulting fermentation products.

The genus Caldicellulosiruptor includes different species of extremely thermophilic (growth at temperature significantly above 70 degrees centigrade) cellulolytic and hemicellulolytic strictly anaerobic nonsporeforming bacteria. The first bacterium of this genus, Caldicellulosiruptor saccharolyticus strain Tp8T 6331 (DSM 8903) has a temperature optimum of 70 degrees centigrade and was isolated from a thermal spring in New Zealand (Rainey et al., 1994; Sissons et al., 1987). It hydrolyses a variety of polymeric carbohydrates with the production of acetate, lactate and trace amounts of ethanol (Donnison et al., 1988). Phylogenetic analysis showed that it constitutes a novel lineage within the Bacillus/Clostridium subphylum of the Gram-positive bacteria (Rainey et al. 1994).

According to the present disclosure, the microorganisms produce lactic acid and show several features that distinguish them from currently used microorganisms: (i) high yield and low product inhibition, (ii) simultaneous utilization of lignocellolytic biomass material and/or starch, and (iii) growth at elevated temperatures. The microorganisms according to the present disclosure are robust thermophile organisms with a decreased risk of contamination. They efficiently convert an extraordinarily wide range of biomass components to carbon-based chemicals like lactic acid.

In an advantageous embodiment, the microorganism used in the fermentation process according to the present disclosure is Caldicellulosiruptor sp. BluConL60, or a microorganism derived therefrom, mutants or a homolog thereof, in particular of mutants thereof retaining the properties of the BluConL60 strain. The BluConL60 microorganism was deposited on Aug. 29, 2019 under the accession number DSM 33252 according to the requirements of the Budapest Treaty at the Deutsche Sammlung von Mikroorganismen and Zellkulturen (DSMZ), Inhoffenstraße 7B, 38124 Braunschweig (DE) by BluCon Biotech GmbH, Nattermannallee 1, 50829 Cologne (DE).

As used herein “mutant” or “homolog” means a microorganism derived from the cells or strains according to the present disclosure, which are altered due to a mutation. A mutation is a change produced in cellular DNA, which can be spontaneous, caused by an environmental factor or errors in DNA replication, or induced by physical or chemical conditions. The processes of mutation included in this and indented subclasses are processes directed to production of essentially random changes to the DNA of the microorganism including incorporation of exogenous DNA. All mutants of the microorganisms comprise the advantages of being extreme thermophile (growing and fermenting at temperatures above 70 degrees centigrade) and are capable of fermenting starchy and lignocellulosic biomass to lactic acid, in particular to L-lactic acid. In an advantageous embodiment, mutants of the microorganisms according to the present disclosure have in a DNA-D NA hybridization assay, a DNA-DNA relatedness of at least 80%, preferably at least 90%, at least 95%, more preferred at least 98%, most preferred at least 99%, and most preferred at least 99.9% with the isolated bacterial strain of Caldicellulosiruptor species. In particular, the mutants of Caldicellulosiruptor species retaining the properties of the deposited strain of Caldicellulosiruptor species.

The Caldicellulosiruptor sp. strains according to the present disclosure have several highly advantageous characteristics needed for the conversion of unmodified starch containing biomass/material. Thus, these base strains possess all the genetic machinery for the hydrolysis of starch, cellulose and hemicelluloses and for the conversion of both pentose and hexose sugars to various fermentation products such as lactic acid. As will be apparent from the below examples, the examination of the complete 16S rDNA sequence showed that the closely related strains may all be related to Caldicellulosiruptor saccharolyticus although the 16S rDNA sequences may place them in a separate subspecies or even a different species

In the processes for the production of lactic acid according to the present disclosure, the isolated bacterial strains Caldicellulosiruptor sp. DIB004C, DIB041C, DIB087C, DIB101C, DIB103C, DIB104C and DIB107C can in particular be used for the conversion of the unmodified starch and/or unmodified starch containing biomass/material.

TABLE 1
Strains of Caldicellulosiruptor used for unmodified starch conversion to lactic acid

| | | | DSMZ | | |
|---|---|---|---|---|---|
| | Spe- | | accession | Deposition | |
| Genus | cies | Name | number | date | Depositor |
| Caldicellulosiruptor | sp. | DIB004C | DSM 25177 | Sep. 15, 2011 | Direvo Industrial |
| | | | | | Biotechnology |
| | | | | | GmbH |
| Caldicellulosiruptor | sp. | DIB041C | DSM 25771 | Mar. 15, 2012 | Direvo Industrial |
| | | | | | Biotechnology |
| | | | | | GmbH |
| Caldicellulosiruptor | sp. | DIB087C | DSM 25772 | Mar. 15, 2012 | Direvo Industrial |
| | | | | | Biotechnology |
| | | | | | GmbH |
| Caldicellulosiruptor | sp. | DIB101C | DSM 25178 | Sep. 15, 2011 | Direvo Industrial |
| | | | | | Biotechnology |
| | | | | | GmbH |
| Caldicellulosiruptor | sp. | DIB103C | DSM 25773 | Mar. 15, 2012 | Direvo Industrial |
| | | | | | Biotechnology |
| | | | | | GmbH |
| Caldicellulosiruptor | sp. | DIB104C | DSM 25774 | Mar. 15, 2012 | Direvo Industrial |
| | | | | | Biotechnology |
| | | | | | GmbH |
| Caldicellulosiruptor | sp. | DIB107C | DSM 25775 | Mar. 15, 2012 | Direvo Industrial |
| | | | | | Biotechnology |
| | | | | | GmbH |
| Caldicellulosiruptor | sp | BluCon006 | DSM 33095 | Apr. 9, 2019 | BluCon Biotech |
| | | | | | GmbH |
| Caldicellulosiruptor | sp | BluCon014 | DSM 33096 | Apr. 9, 2019 | BluCon Biotech |
| | | | | | GmbH |
| Caldicellulosiruptor | sp | BluCon016 | DSM 33097 | Apr. 9, 2019 | BluCon Biotech |
| | | | | | GmbH |
| Caldicellulosiruptor | sp | BluConL60 | DSM 33252 | Aug. 29, 2019 | BluCon Biotech |
| | | | | | GmbH |

The strains listed in Table 1 have been deposited in accordance with the terms of the Budapest Treaty with DSMZ—Deutsche Sammlung von Mikroorganismen und Zellkulturen GmbH, Inhoffenstr. 7B, 38124 Braunschweig, Germany, under the respectively indicated DSMZ accession numbers and deposition dates.

In a preferred embodiment, the Caldicellulosiruptor sp. microorganism is

a) Caldicellulosiruptor sp. strain BluConL60 that was deposited on Aug. 29, 2019 under the accession number DSM 33252 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE),

b) a microorganism derived from Caldicellulosiruptor sp. BluConL60 or

c) a Caldicellulosiruptor sp. BluConL60 mutant retaining the properties of BluConL60.

All strains and mutant thereof and in table 1 belong to the genus Caldicellulosiruptor and are strictly anaerobic, non-sporeforming, non-motile, gram-positive bacteria. Cells are straight rods 0.4-0.5 μm by 2.0-4.0 μm, occurring both singly and in pairs. After 7 days incubation at 72 degrees centigrade on solid medium with agar and cellulose as substrate both strains form circular milky colonies of 0.5-1 mm in diameter. Clearing zones around the colonies are produced indicating cellulose degradation.

The term “a microorganism” as used herein may refer to only one unicellular organism as well as to numerous single unicellular organisms. For example, the term “a microorganism of the genus Caldicellulosiruptor” may refer to one single Caldicellulosiruptor bacterial cell of the genus Caldicellulosiruptor as well as to multiple bacterial cells of the genus Caldicellulosiruptor.

The terms “a strain of the genus Caldicellulosiruptor” and “a Caldicellulosiruptor cell” are used synonymously herein. In general, the term “a microorganism” refers to numerous cells. In particular, said term refers to at least 10³ cells, preferably at least 10⁴ cells, at least 10⁵ or at least 10⁶ cells.

As mentioned above starch containing biomass according to the present disclosure can be but is not limited corn (maize), wheat, oats, rice, potato, peas, cassava, and starchy biomass material obtained through processing of food plants. In advantageous embodiments, the starch containing biomass material is starchy biomass material through processing of food plants, preferably processed peas.

In advantageous embodiments, the starch-containing biomass is starch-containing plant material, including: tubers, roots, whole grain; and any combination thereof. The starch-containing biomass/material may be obtained from cereals. Suitable starch-containing biomass/material includes corn (maize), wheat, barley, cassava, sorghum, rye, potato, peas or any combination thereof. Peas is the preferred feedstock. The starch-containing material may also consist of or comprise, e.g., a side stream from starch processing, e.g., C6 carbohydrate containing process streams that may not be suited for production of syrups. Whole stillage typically contains about 10-15 wt-% dry solids. Whole stillage components include fiber, hull, germ, oil and protein components from the starch-containing feedstock as well as non-fermented starch.

In advantageous embodiments the cells, strains, microorganisms may be modified in order to obtain mutants or derivatives with improved characteristics. Thus, in one embodiment there is provided a bacterial strain according to the disclosure, wherein one or more genes have been inserted, deleted or substantially inactivated. The variant or mutant is typically capable of growing in a medium comprising a starch containing biomass material and/or lignocellulosic biomass material.

In another embodiment, there is provided a process for preparing variants or mutants of the microorganisms according to the present disclosure, wherein one or more genes are inserted, deleted or substantially inactivated as described herein.

In some embodiments, one or more additional genes are inserting into the strains according to the present disclosure. Thus, in order to improve the yield of the specific fermentation product, it may be beneficial to insert one or more genes encoding a polysaccharase into the strain according to the invention. Hence, in specific embodiments there is provided a strain and a process according to the invention wherein one or more genes encoding a polysaccharase which is selected from cellulases (such as EC 3.2.1.4); beta-glucanases, including glucan-1,3 beta-glucosidases (exo-1,3 beta-glucanases, such as EC 3.2.1.58), 1,4-beta-cellobiohydrolases (such as EC 3.2.1.91) and endo-1,3(4)-beta-glucanases (such as EC 3.2.1.6); xylanases, including endo-1,4-beta-xylanases (such as EC 3.2.1.8) and xylan 1,4-beta-xylosidases (such as EC 3.2.1.37); pectinases (such as EC 3.2.1.15); alpha-glucuronidases, alpha-L-arabinofuranosidases (such as EC 3.2.1.55), acetylesterases (such as EC 3.1.1.-), acetylxylanesterases (such as EC 3.1.1.72), alpha-amylases (such as EC 3.2.1.1), beta-amylases (such as EC 3.2.1.2), glucoamylases (such as EC 3.2.1.3), pullulanases (such as EC 3.2.1.41), beta-glucanases (such as EC 3.2.1.73), hemicellulases, arabinosidases, mannanases including mannan endo-1,4-beta-mannosidases (such as EC 3.2.1.78) and mannan endo-1,6-alpha-mannosidases (such as EC 3.2.1.101), pectin hydrolases, polygalacturonases (such as EC 3.2.1.15), exopolygalacturonases (such as EC 3.2.1.67) and pectate lyases (such as EC 4.2.2.10), are inserted.

In accordance with the present disclosure, a method of producing a fermentation product comprising culturing a strain according to the invention under suitable conditions is also provided.

The strains according to the disclosure are strictly anaerobic microorganisms, and hence it is preferred that the fermentation product is produced by a fermentation process performed under strictly anaerobic conditions. Additionally, the strain according to invention is an extremely thermophilic microorganism, and therefore the process may perform optimally, when it is operated at temperature in the range of about 40-95 degrees centigrade, such as the range of about 50-90 degrees centigrade, including the range of about 60-85 degrees centigrade, such as the range of about 65-75 degrees centigrade

For the production of lactic acid, it may be useful to select a specific fermentation process, such as batch fermentation process, including a fed-batch process or a continuous fermentation process. Also, it may be useful to select a fermentation reactor such as a stirred vessel reactor, an immobilized cell reactor, a fluidized bed reactor or a membrane bioreactor.

In accordance with the invention, the method is useful for the production of lactic acid, the enantiomers L-lactic acid and D-lactic acid and the racemic compound D/L-lactic acid.

The fermentation conditions to form lactic acid and/or lactate are known per se and are described in WO 01/27064, WO 99/19290, and WO 98/15517. Accordingly, the temperature may range from 0 to 80° C., while the pH (which decreases upon lactic acid formation) ranges from 3 to 8. A pH below 5 is generally desirable, as part of the lactic acid formed will then be present in its free-acid form instead of in its salt form. Furthermore, at low pH there is less risk of contamination with other micro organisms. Any of the many known types of apparatus may be used for the fermentation according to the present invention.

The microorganism according to the present invention may be used as a biologically pure culture or it may be used with other lactic acid producing microorganisms in mixed culture. Biologically pure cultures are generally easier to optimize but mixed cultures may be able to utilize additional substrates. One may also add enzyme (s) to the fermentation vessel to aid in the degradation of substrates or to enhance lactic acid production. For example, cellulase may be added to degrade cellulose to glucose simultaneously with the fermentation of glucose to lactic acid by microorganisms. Likewise, a hemicellulase may be added to degrade hemicellulose. As mentioned-above, said hydrolyzation (optionally by means of enzymes) may also be conducted prior to fermentation.

The thermophilic Caldicellulosiruptor species-containing fermentation broth cultures used in the processes according to the present disclosure are relatively resistant to contamination by other microorganisms.

The thermophilic Caldicellulosiruptor species used in the process according to the disclosure may be grown both in so-called chemically defined media and in culture media which contain undefined compounds such as yeast extracts, peptone, tryptone, meat extract and other complex nitrogen sources. The use of a chemically defined medium is preferred because it results in lactic acid and/or lactate with less impurities.

After fermentation, the lactic acid and/or lactate is separated from the fermentation broth by any of the many conventional techniques known to separate lactic acid and/or lactate from aqueous solutions. Particles of substrate or microorganisms (the biomass) may be removed before separation to enhance separation efficiency. Said separation may be conducted by means of centrifuging, filtration, flocculation, flotation or membrane filtration. This is for instance known from WO 01/38283 wherein a continuous process for the preparation of lactic acid by means of fermentation is described. While the discussion of the fermentation in this specification generally refers to a batch process, parts or all of the entire process may be performed continuously. To retain the microorganisms in the fermentor, one may separate solid particles from the fermentation fluids. Alternatively, the microorganisms may be immobilized for retention in the fermentor or to provide easier separation.

After separation of the lactic acid and/or lactate from the fermentation broth, the product may be subjected to one or more purification steps such as extraction, distillation, crystallization, filtration, treatment with activated carbon etcetera. The various residual streams may be recycled, optionally after treatment, to the fermentation vessel or to any previously performed purification step.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”.

The following methods and examples are offered for illustrative purposes only, and are not intended to limit the scope of the present disclosure in any way

### Methods and Examples

In the following examples, materials and methods of the present disclosure are provided including the determination of the properties of the microbial strains according to the present disclosure. It should be understood that these examples are for illustrative purpose only and are not to be construed as limiting this disclosure in any manner.

Description of Caldicellulosiruptor sp. Strain BluConL60

Caldicellulosiruptor sp strain BluConL60 listed in Table 1 was deposited on Aug. 29, 2019 under the accession number DSM 33252 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Description of Caldicellulosiruptor sp. Strain BluCon006, Caldicellulosiruptor sp. Strain BluCon014 and Caldicellulosiruptor sp. Strain BluCon016

Caldicellulosiruptor sp. BluCon006, Caldicellulosiruptor sp. BluCon014 and Caldicellulosiruptor sp. BluCon016, which are listed in Table 2, are deposited on Apr. 9, 2019 under the accession numbers DSM 33095, DSM 33096 and DSM 33097 according to the requirements of the Budapest Treaty at the Deutsche Sammlung von Mikroorganismen und Zellkulturen (DSMZ), Inhoffenstraße 7B, 38124 Braunschweig (DE) by BluCon Biotech GmbH, Nattermannallee 1, 50829 Cologne (DE).

Description of Caldicellulosiruptor sp. DIB104C

Caldicellulosiruptor sp. DIB104C listed in Table 2 was deposited on Mar. 15, 2012 under the accession number DSM 25774 according to the requirements of the Budapest Treaty at the Deutsche Sammlung von Mikroorganismen und Zellkulturen (DSMZ), Inhoffenstraße 7B, 38124 Braunschweig (DE) by DIREVO Industrial Biotechnology GmbH, Nattermannallee 1, 50829 Cologne (DE).

TABLE 2
Survey of Description of Caldicellulosiruptor sp

| | | | DSMZ | |
|---|---|---|---|---|
| | Spe- | | accession | Deposition |
| Genus | cies | Name | number | date |
| Caldicellulosiruptor | sp | DIB104C | DSM 25774 | Mar. 15, 2012 |
| Caldicellulosiruptor | sp | BluCon006 | DSM 33095 | Apr. 9, 2019 |
| Caldicellulosiruptor | sp | BluCon014 | DSM 33096 | Apr. 9, 2019 |
| Caldicellulosiruptor | sp | BluCon016 | DSM 33097 | Apr. 9, 2019 |
| Caldicellulosiruptor | sp | BluConL60 | DSM 33252 | Aug. 29, 2019 |

### Example 1: HPLC

Sugars and fermentation products were quantified by HPLC-RI using a Prominence LC-20AD HPLC (company Shimadzu) fitted with a Rezex ROA Organic Acid H+ (Phenomenex). The analytes were separated isocratically with 2.5 mM H₂SO₄ and at 65 degrees centigrade.

### Example 2: Cultivation of Seed Culture

All procedures for enrichment and isolation of the strains listed in Table 2 employed anaerobic technique for strictly anaerobic bacteria (Hungate 1969). The strains listed in Table 2 were cultivated at 70 degrees centigrade with filter paper (i.e. cellulose) as substrate in seed medium. The cells were cultured under strictly anaerobic conditions applying the following basic medium:

Seed medium

| Filter paper Whatman#1 (of the size of | Approx.. 5.0 | g |
|---|---|---|
| 1 × 6 cm (approx. 50 mg) was added) | | |
| D-glucose, water free | 0.5 | g |
| NH₄Cl | 1.0 | g |
| NaCl | 0.5 | g |
| MgSO₄ × 7 H₂O | 0.3 | g |
| CaCl₂ × 2 H₂O | 0.05 | g |
| NaHCO₃ | 0.5 | g |
| K₂HPO₄ | 1.5 | g |
| KH₂PO₄ | 3.0 | g |
| Yeast extract (BD) | 0.5 | g |
| Trace elements stock solution | 0.5 | ml |
| Resazurin, Na-salt | 0.25 | mg |
| L-cysteine | 0.5 | g |
| Distilled water | 1000 | ml |

Trace elements stock solution

| | | |
|---|---|---|
| NiCl₂ × 6H₂O | 2 | g |
| FeSO₄ × 7H₂O | 1 | g |
| NH₄Fe(III) citrate, 18% Fe | 10 | g |
| MnSO₄ × H₂O | 5 | g |
| CoCl₂ × 6H₂O | 1 | g |
| ZnSO₄ × 7H₂O | 1 | g |
| CuSO₄ × 5H₂O | 0.1 | g |
| H₃BO₃ | 0.1 | g |
| Na₂MoO₄ × 2H₂O | 0.1 | g |
| Na₂SeO₃ | 0.2 | g |
| Na₂WoO₄ × 2H₂O | 0.13 | g |
| Distilled water | 1000 | ml |

Add 0.5 ml of the trace elements stock solution to 1 liter of the medium

Cultivation was performed in 16 ml total volume in Hungate tubes, with butyl rubber stoppers and screw caps. Into each tube one strip of filter paper, Whatman #1 of the size of 1×6 cm (approx. 50 mg) had been added. The tubes containing the filter paper were flushed with nitrogen gas (purity 99.999%); closed with rubber stoppers and incubated for 60 minutes to remove oxygen from paper.

In seed medium B all ingredients except L-cysteine are dissolved in deionized water and the medium was flushed with nitrogen gas (purity 99.999%) for 20 min at room temperature and the pH-value was adjusted to 7.0 at room temperature with 5 M NaOH. Then a sterile stock solution of L-cysteine, which had been filtered into a nitrogen containing serum flasks, was added to the medium. The medium was then dispensed into serum flasks under nitrogen atmosphere and the vessels were tightly sealed. After autoclaving at 121 degrees centigrade for 20 min pH-value should be in between 7.0 and 7.2.

Subsequent to autoclaving, cultures were inoculated by injection of the seed culture through the seal septum and inoculated in an incubator at 70 degrees centigrade for 24 to 48 hours.

### Example 3: Fermentation with Starch (Soluble)

Batch experiments with strains listed in Table 2 were performed by cultivation on the following fermentation medium with addition of 90 g/L of starch (soluble) and 10 to 100 g/l CaCO₃:

Fermentation medium

| CaCO3 | 10 to 100 | g |
|---|---|---|
| Starch (soluble) | 90 | g |
| (NH₄)₂HPO₄ | 0.5 to 1.0 | g |
| (NH₄)₂SO₄ | 2.0 to 5.0 | g |
| NaCl | 0.1 to 0.5 | g |
| MgSO₄ × 7 H₂O | 0.2 to 0.8 | g |
| CaCl₂ × 2 H₂O | 0.05 to 0.5 | g |
| KH₂PO₄ | 0.2 to 0.5 | g |
| Yeast extract | 1.0 to 6.0 | g |
| Meat extract | 2.0 to 10.0 | g |
| Corn steep liquor | 1.0 to 5.0 | g |
| Peptone | 5.0 to 10 | g |
| Trace elements stock solution | 0.5 to 1.0 | ml |
| Vitamin stock solution | 1.0 to 4.0 | ml |
| Resazurin, Na-salt | 0.25 | mg |
| L-cysteine | 0.5 | g |
| Distilled water | 1000 | ml |

Vitamin stock solution

| | | |
|---|---|---|
| Nicotinic acid | 1000 | mg |
| Cyanocobalamin (B12) | 125 | mg |
| p-Aminobenzoic acid (4-aminobenzoic acid) | 125 | mg |
| Calcium D-pantothenate | 125 | mg |
| Thiamine-HCl | 125 | mg |
| Riboflavin (B2) | 125 | mg |
| Lipoic acid | 125 | mg |
| Folic acid | 50 | mg |
| Biotin (vitamin H) | 50 | mg |
| Pyridoxin-HCl (B6) | 50 | mg |
| Distilled water | 1000 | ml |

Trace elements stock solution

| | | |
|---|---|---|
| NiCl₂ × 6H₂O | 2 | g |
| FeSO₄ × 7H₂O | 1 | g |
| NH₄Fe(III) citrate, 18% Fe | 10 | g |
| MnSO₄ × H₂O | 5 | g |
| CoCl₂ × 6H₂O | 1 | g |
| ZnSO₄ × 7H₂O | 1 | g |
| CuSO₄ × 5H₂O | 0.1 | g |
| H₃BO₃ | 0.1 | g |
| Na₂MoO₄ × 2H₂O | 0.1 | g |
| Na₂SeO₃ | 0.2 | g |
| Na₂WoO₄ × 2H₂O | 0.13 | g |
| Distilled water | 1000 | ml |

Add 0.5 ml of the trace elements stock solution to 1 liter of the medium

Cultivation was performed in 100 ml serum bottles closed with butyl rubber stoppers and aluminum crimp seals. All ingredients except L-cysteine were dissolved in deionized water and the medium was flushed with nitrogen gas (purity 99.999%) for 20 min at room temperature. Then a sterile stock solution of L-cysteine, which had been filtered into a nitrogen containing serum flasks, was added to the medium. The medium was then dispensed into serum flasks under nitrogen atmosphere and the vessels were tightly sealed. After autoclaving at 121 degrees centigrade for 20 min pH-value should be in between 7.1 and 7.3. Duplicate cultivations were started by addition of seed culture prepared as described in example 2. The cultures were incubated at 72 degrees centigrade for four days. Samples were taken and sugars and fermentation products were quantified by HPLC analysis as described in example 1. The results are presented in Table 3 and indicate efficient production of lactic acid from starch.

TABLE 3
Lactic acid (average of two fermentations) from soluble
starch by different microorganisms of the genus Caldicellulosiruptor
after 4 days cultivation.

| | Lactic acid |
|---|---|
| Strain | [g/l] |
| BluConL60 | 43.8 ± 1.2 |
| DIB104C | 13.0 ± 0.3 |
| BluCon006 | 29.1 ± 0.4 |
| BluCon014 | 23.1 ± 0.5 |
| BluCon016 | 22.9 ± 0.6 |

### Example 4: Cultivation of Seed Culture

All procedures for enrichment and isolation of BluConL60 employed anaerobic technique for strictly anaerobic bacteria (Hungate 1969). BluConL60 was cultivated at 70 degree centigrade with crystalline cellulose as substrate in seed medium. The cells were cultured under strictly anaerobic conditions applying the following seed medium:

Seed medium

| | | |
|---|---|---|
| Crystalline cellulose (Avicel pH 101) | 10 | g |
| NH₄Cl | 1.0 | g |
| NaCl | 0.5 | g |
| MgSO₄ × 7 H2O | 0.3 | g |
| CaCl₂ × 2 H₂O | 0.05 | g |
| NaHCO₃ | 0.5 | g |
| K₂HPO4 | 1.5 | g |
| KH₂PO₄ | 3.0 | g |
| Yeast extract (BD) | 0.5 | g |
| Trace elements stock solution | 0.5 | ml |
| Resazurin, Na-salt | 0.25 | mg |
| L-cysteine | 0.5 | g |
| Distilled water | 1000 | ml |

Trace elements stock solution

| | | |
|---|---|---|
| NiCl₂ × 6H₂O | 2 | g |
| FeSO₄ × 7H₂O | 1 | g |
| NH₄Fe(III) citrate, 18% Fe | 10 | g |
| MnSO₄ × H₂O | 5 | g |
| CoCl₂ × 6H₂O | 1 | g |
| ZnSO₄ × 7H₂O | 1 | g |
| CuSO₄ × 5H₂O | 0.1 | g |
| H₃BO₃ | 0.1 | g |
| Na₂MoO₄ × 2H₂O | 0.1 | g |
| Na₂SeO₃ | 0.2 | g |
| Na₂WoO₄ × 2H₂O | 0.13 | g |
| Distilled water | 1000 | ml |

All ingredients except L-cysteine were dissolved in deionized water and the medium was flushed with nitrogen gas (purity 99.999%) for 20 min at room temperature and the pH-value was adjusted to 7.0 at room temperature with 5 M NaOH. The medium was then dispensed into serum flasks under nitrogen atmosphere and the vessels are tightly sealed. After autoclaving at 121° C. for 20 min pH-value should be in between 7.0 and 7.2.

Subsequent to autoclaving, cultures were inoculated by injection of the seed culture through the seal septum and inoculated in an incubator at 70° C. for 16 to 48 hours.

### Example 5: Lactate Biosensor

L-lactic acid concentration of the samples were quantified by the lactate biosensor LaboTRACE compact (company TRACE Analytics GmbH, Braunschweig, Germany) according to the instructions of the company.

### Example 6: Fermentation with Unmodified Starch (Pure Potato Starch)

Batch experiments with strain BluConL60 were performed in cultivations in the following fermentation medium.

Fermentation medium

| CaCO₃ | 10 to 100 | g |
|---|---|---|
| Unmodified starch (pure potato starch) | 50 | g |
| (NH₄)₂HPO₄ | 0.5 to 1.0 | g |
| (NH₄)₂SO₄ | 2.0 to 5.0 | g |
| NaCl | 0.1 to 0.5 | g |
| MgSO₄ × 7 H₂O | 0.2 to 0.8 | g |
| CaCl₂ × 2 H₂O | 0.05 to 0.3 | g |
| KH₂PO₄ | 0.2 to 0.5 | g |
| Yeast extract | 1.0 to 6.0 | g |
| Meat extract | 2.0 to 10.0 | g |
| Corn steep liquor | 1.0 to 5.0 | g |
| Peptone | 5.0 to 10 | g |
| Trace elements stock solution | 0.5 to 1.0 | ml |
| Resazurin, Na-salt | 0.25 | mg |
| L-cysteine | 0.5 | g |
| Distilled water | 1000 | ml |

All ingredients except for unmodified starch and L-cysteine were added and dissolved (except for CaCO₃) in deionized water and added into 2 L fermentation vessels with stirrers and pH and temperature control (company BBI-Biotech, Berlin). The pH-value should be between 6.8 and 7.0.

After autoclaving at 121 degree centigrade for 20 min and cooling down to room temperature (25 to 30 degrees centigrade) 50 g/L of unmodified pure potato starch (brand name Küchenmeister, company Frießinger Mühle, Bad Wimpfen), was added and the medium was flushed with nitrogen gas (purity 99.999%) for 20 min at room temperature to remove excess oxygen before L-cysteine, which was dissolved as a stock solution in deionized water (100 g/L), and which had been filtered into a nitrogen containing serum flasks, was added to the medium. Then the fermentation was started by addition of the seed culture. Fermentation batch experiment was carried out in duplicate.

Then the temperature was increased from room temperature to 62 to 75 degree centigrade and the fermentation temperature during the process was regulated between these temperature ranges. PH-value was regulated from 5.8 to 7.2 after 18 h after the fermentation process had started by a solution of Ca(OH)₂ and NH₄OH. Samples were taken and L-lactic acid concentrations were by determined by Lactate biosensor as described in example 5. The results are presented in Table 4.

TABLE 4
L-lactic acid concentrations (average of two fermentations
and deviation from average) from unmodified starch (pure
potato starch) by BluConL60 at different cultivation times.

| Cultivation time [h] | L-lactic acid [g/l] |
|---|---|
| 0.1 | 0.2 ± 0.1 |
| 19 | 10.6 ± 0.4 |
| 25 | 30.5 ± 0.2 |

The results of the fermentation samples show that BluConL60 produces L-lactic acid from unmodified starch.

### Example 7: Enzymatic Quantification of L-Lactic Acid and D-Lactic Acid

### Enzymatic Determination of L-Lactic Acid and D-Lactic Acid Concentrations

In addition to quantification of HPLC sugars and fermentation products like lactic acid the enantiomers L-lactic acid and D-lactic acid concentrations of the final fermentation samples were quantified using the D-/L-Lactic Acid (D-/L-Lactate) (Rapid) Assay Kit K-DLATE (company Megazyme Ltd., Bray Business Park, Bray, Co. Wicklow, A98 YV29, Ireland) according to the instructions of the company.

### Calculation of the Ratio of L-Lactic Acid and D-Lactic to Total Lactic Acid in Percent [%]

Total lactic acid concentrations were calculated by adding up the numbers of L-lactic acid and D-lactic acid concentrations quantified by using the D-/L-Lactic Acid Assay Kit K-DLATE.

The ratio of L-lactic acid and D-lactic on total lactic acid were calculated by dividing each L-lactic acid and D-lactic concentration by total lactic acid concentrations. The obtained numbers were then multiplied by 100 to obtain the results in percent [%].

### Example 8: Fermentation with Unmodified Pure Potato Starch

Fermentation with strain BluConL60 was performed in the following fermentation medium.

Fermentation medium

| CaCO₃ | 10 to 100 | g |
|---|---|---|
| Unmodified starch (pure potato starch) | 50 | g |
| (NH₄)₂HPO₄ | 0.5 to 1.0 | g |
| (NH₄)₂SO₄ | 2.0 to 5.0 | g |
| NaCl | 0.1 to 0.5 | g |
| MgSO₄ × 7 H₂O | 0.2 to 0.8 | g |
| CaCl₂ × 2 H₂O | 0.05 to 0.3 | g |
| KH₂PO₄ | 0.2 to 0.5 | g |
| Yeast extract | 1.0 to 6.0 | g |
| Meat extract | 2.0 to 10.0 | g |
| Corn steep liquor | 1.0 to 5.0 | g |
| Peptone | 5.0 to 10 | g |
| Trace elements stock solution | 0.5 to 1.0 | ml |
| Resazurin, Na-salt | 0.25 | mg |
| L-cysteine | 0.5 | g |
| Distilled water | 1000 | ml |

All ingredients except for unmodified starch and L-cysteine were added and dissolved (except for CaCO₃) in deionized water. 1.46 L of the fermentation medium was added into a 2 L fermentation vessel with stirrers and pH and temperature control (company bbi-biotech, Berlin). The pH-value should be between 6.8 and 7.0.

After autoclaving at 121 degree centigrade for 20 min and cooling down to room temperature (25 to 30 degrees centigrade) 50 g/L of unmodified pure potato starch (brand name Küchenmeister, company Frießinger Mühle, Bad Wimpfen), was added and the medium was flushed with nitrogen gas (purity 99.999%) for 20 min at room temperature to remove excess oxygen before L-cysteine, which was dissolved as a stock solution in deionized water (100 g/L), and which had been filtered into a nitrogen containing serum flasks, was added to the medium. Then the fermentation was started by addition of 100 mL of the seed culture (see: Example 4).

Then the temperature was increased from room temperature to 62 to 75 degree centigrade and the fermentation temperature during the process was regulated between these temperature ranges. PH-value was regulated from 5.8 to 7.2 after 18 h after the fermentation process had started by a solution of Ca(OH)₂ and NH₄OH.

After 26 h and 48 h after the fermentation process has started 75 g of unmodified pure potato starch was added to the cultivation.

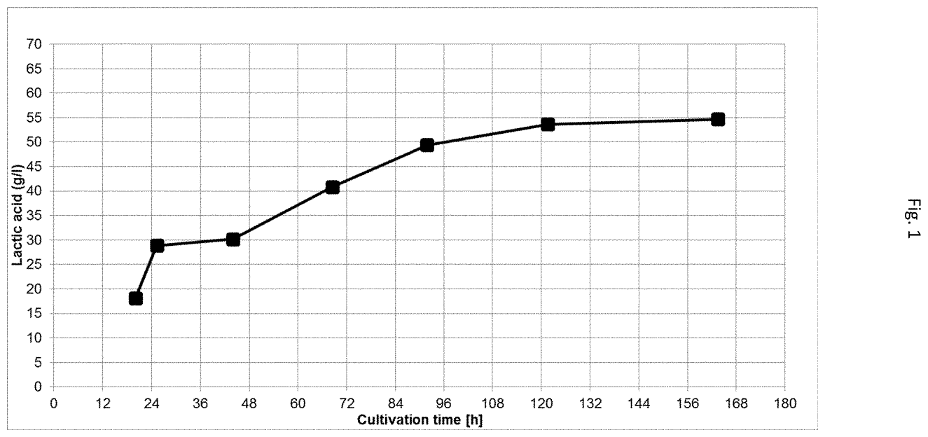

Samples were taken and lactic acid concentrations were by determined by HPLC analysis as described in Example 1. The results are presented in FIG. 1.

The final sample, which had been analyzed according to Example 7, contained 99.6% L-lactic acid and 0.4% D-lactic acid.

The results of the fermentation samples show that BluConL60 produces L-lactic acid and traces of D-lactic acid from unmodified pure potato starch.

### Example 9: Fermentation with Unmodified Pea Starch

Fermentation with strain BluConL60 was performed in the fermentation medium and in a process according to Example 8 except that unmodified pea starch was used.

After 26 h and 48 h after the fermentation process has started 75 g of unmodified pea starch was added to the cultivation.

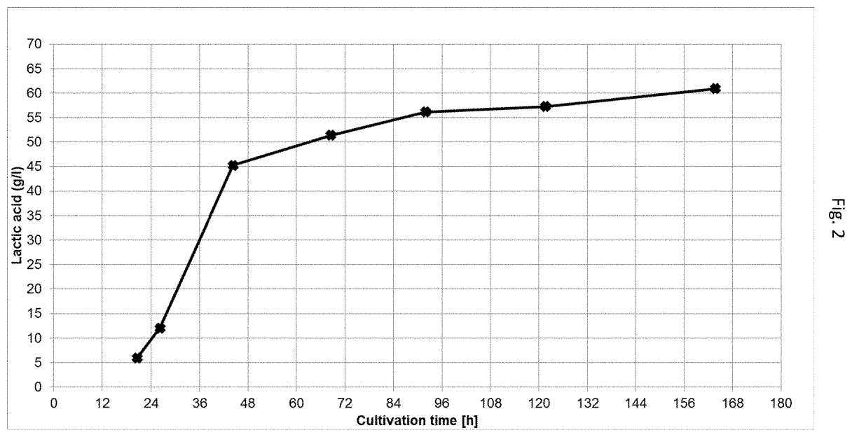

Samples were taken and lactic acid concentrations were by determined by HPLC analysis as described in Example 1. The results are presented in FIG. 2.

The final sample, which had been analyzed according to Example 7, contained 99.3% L-lactic acid and 0.7% D-lactic acid.

The results of the fermentation samples show that BluConL60 produces L-lactic acid and traces of D-lactic acid from unmodified pea starch.

### Example 10: Fermentation with Unmodified Wheat Starch

Fermentation with strain BluConL60 was performed in the fermentation medium and in a process according to Example 8 except that unmodified wheat starch was used.

After 26 h and 50 h after the fermentation process has started 75 g of unmodified wheat starch was added to the cultivation.

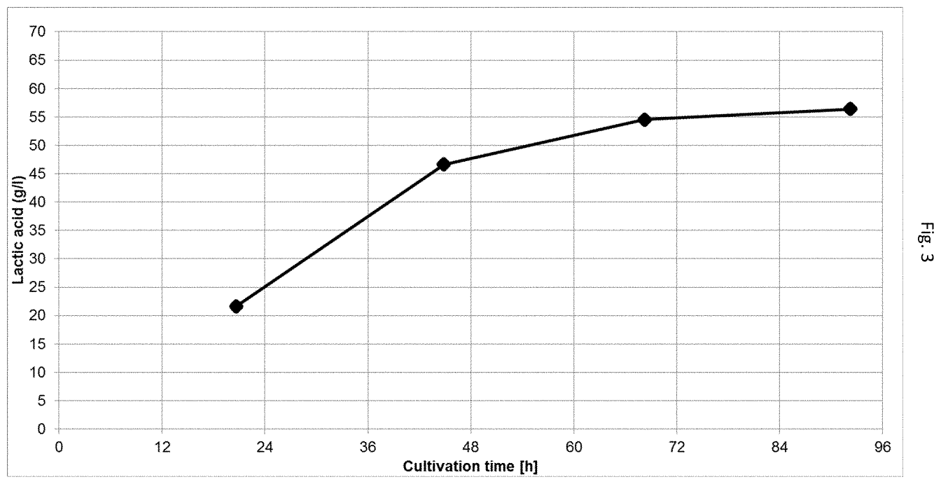

Samples were taken and lactic acid concentrations were by determined by HPLC analysis as described in Example 1. The results are presented in FIG. 3.

The final sample, which had been analyzed according to Example 7, contained 99.6% L-lactic acid and 0.4% D-lactic acid.

The results of the fermentation samples show that BluConL60 produces L-lactic acid and traces of D-lactic acid from unmodified wheat starch.

### Example 11: Fermentation with Unmodified Tapioca Starch

Fermentation with strain BluConL60 was performed in the fermentation medium and in a process according to Example 8 except that unmodified wheat starch was used.

After 26 h and 50 h after the fermentation process has started 75 g of unmodified tapioca (cassava) starch was added to the cultivation.

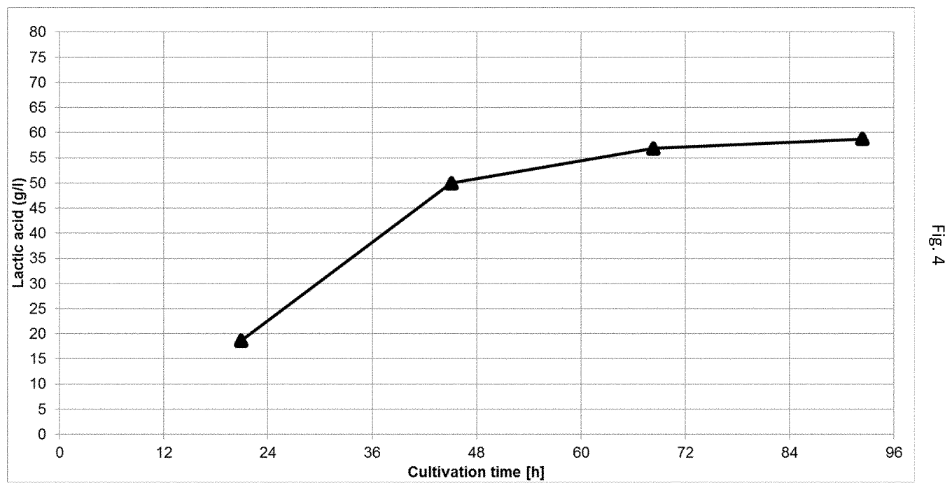

Samples were taken and lactic acid concentrations were by determined by HPLC analysis as described in Example 1. The results are presented in FIG. 4.

The final sample, which had been analyzed according to Example 7, contained 99.6% L-lactic acid and 0.4% D-lactic acid.

The results of the fermentation samples show that BluConL60 produces L-lactic acid and traces of D-lactic acid from unmodified tapioca (cassava) starch.

### Example 12: Fermentation with Unmodified Corn (Maize) Starch

Fermentation with strain BluConL60 was performed in the fermentation medium and in a process according to Example 8 except that unmodified wheat starch was used.

After 26 h and 74.5 h after the fermentation process has started 75 g of corn (maize) starch was added to the cultivation.

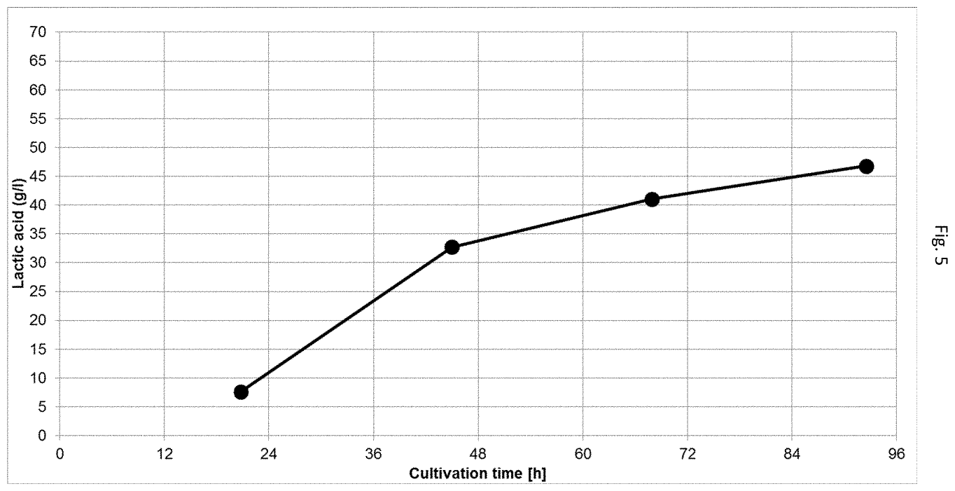

Samples were taken and lactic acid concentrations were by determined by HPLC analysis as described in Example 1. The results are presented in FIG. 5.

The final sample, which had been analyzed according to Example 7, contained 99.8% L-lactic acid and 0.2% D-lactic acid.

The results of the fermentation samples show that BluConL60 produces L-lactic acid and traces of D-lactic acid from unmodified corn (maize) starch.

### LIST OF ADDITIONAL REFERENCES

Karmakar R.; Ban D. and Ghosh. 2014. Comparative study of native and modified starches isolated from conventional and nonconventional sources. International Food Research Journal. 21(2). 597-602.
Bhanwar, S. and Ganguli, A. 2014. Amylase and galactosidase production on potato starch waste by Lactococcus lactis subsp. lactic isolated from pickled yam. Journal of Scientific and Industrial Research 73: 324-330.
Panda H., and Ray R., 2016. Amylolytic Lactic Acid Bacteria Microbiology and Technological Interventions in Food Fermentations. In book: Fermented foods. Part 1. Biochemistry & Biotechnology. 1st Edn. CRC press. Editors: Didier Montet & Ramesh C Ray.
John, R. P., Nampoothiri, M. K. and Pandey, A. 2007. Polyurethane foam as an inert carrier for the production of L(+) lactic acid by Lactobacillus casei under solid state fermentation. Letter in Applied Microbiology 44: 582-587.
Narita, J., Nakahara, S., Fukuda, H. and Kondo, A. 2004. Efficient production of L-(+)-lactic acid from raw starch by Streptococcus bovis 148. Journal of Bioscience Bioengineering 97: 423-425.
Naveena, B. J., Altaf, Md., Bhadrayya, K., Madhavendra, S. S. and Reddy, G. 2004. Production of L(+) lactic acid by Lactobacillus amylophilus GV6 in semi-solid state fermentation using wheat bran. Food Technology and Biotechnology 42: 147-152.
Naveena, B. J., Altaf, Md., Bhadrayya, K., Madhavendra, S. S. and Reddy, G. 2005. Direct fermentation of starch to L (+) lactic acid in SSF by Lactobacillus amylophilus GV6 using wheat bran as support and substrate medium optimization using RSM. Process Biochemistry 40: 681-690.
Narita, J., Okano, K., Kitao, T. and Ishida, S. 2006. Display of alpha-amylase on the surface of Lactobacillus casei cells by use of the PgsA anchor protein, and production of lactic acid from starch. Applied and Environmental Microbiology 72: 269-275.
Reddy, G., Md. Altaf, Naveena, B. J., Venkateshwar, M. and Vijay, K. E. 2008. Amylolytic bacterial lactic acid fermentation: A review. Biotechnology Advances 26: 22-34.
Wang Y., Cai W., Luo J., Qi B., Wan Y. 2019. One step open fermentation for lactic acid production from inedible starchy biomass by thermophilic Bacillus coagulans IPE22. Bioresource Technology. 272. 398-406.
Smerilli M.; Neureiter, M.; Wurz, St.; Haas, C.; Frühauf, S.; and Fuchs W. 2014. Direct fermentation of potato starch and potato residues to lactic acid by Geobacillus stearothermophilus under non-sterile conditions. J Chem Technol Biotechnol.; 90: 648-657.
Drumright R. E., Gruber P R, Henton D E. 2000. Polylactic acid technology. Adv Mater 12:1841-1846.
Narayanan N., Roychoudhury P., Srivastava A. 2004. L(+) Lactic acid fermentation and its product polymerization. Electron J Biotechnol 7:167-179.
Data R., Tsai S., Bonsignore P., Moon S., Frank J. 1995. Technological and economic potential of polylactic acid) and lactic acid derivatives. FEMS Microbiol Rev 16:221-231
Vaidya A., Pandey R., Mudliar S., Suresh Kumar M., Chakrabarti T., Devotta S. 2005. Production and recovery of lactic acid for polylactide—an overview. Crit Rev Environ Sci Technol 35:429-467.
Mars A., Veuskems T., Budde M., von Doeveren P., Lipis St, Bakker R., de Vrije and Claasen P. 2010. Biohydrogen production from untreated and hydrolyzed potato steam peels by the extreme thermophiles Caldicellulosiruptor saccharolyticus and Thermotoga neapolitana. International Journal of Hydrogen Energy. 35.7730-7737.
Rainey F A, Donnison A M, Janssen P H, Saul D, Rodrigo A, Bergquist P L, Daniel R M, Stackebrandt E, Morgan H W. (1994) Description of Caldicellulosiruptor saccharolyticus gen. nov., sp. nov: an obligately anaerobic, extremely thermophilic, cellulolytic bacterium. FEMS Microbiol Lett. 120:263-266.
Sissons C H, Sharrock K R, Daniel R M, Morgan H W. (1987) Isolation of cellulolytic anaerobic extreme thermophiles from New Zealand thermal sites. Appl Environ Microbiol. 53:832-838.
Donnison A M, Brockelsby C M, Morgan H W, Daniel R M. (1989) The degradation of lignocellulosics by extremely thermophilic microorganisms. Biotechnol Bioeng. 33:1495-1499.
Hungate R E. (1969) A roll tube method for cultivation of strict anaerobes. In: Methods in Microbiology Eds. Norris J R and Ribbons D W. pp 118-132. New York: Academic Press.
Chenna R, Sugawara H, Koike T, Lopez R, Gibson T J, Higgins D G, Thompson J D. (2003) Multiple sequence alignment with the Clustal series of programs. Nucleic Acids Res. 13:3497-3500.
Kumar S, Tamura K, Jakobsen I B, Nei M. (2001) MEGA2: molecular evolutionary genetics analysis software. Bioinformatics. 17:1244-1245.

1-27. (canceled)

28. A fermentation process for the production of lactic acid comprising the steps of contacting unmodified starch and/or unmodified starch-containing material with a microbial culture comprising a microorganism of the genus Caldicellulosiruptor for a period of time at an initial temperature and an initial pH, thereby producing an amount of a lactic acid, wherein the unmodified starch and/or the unmodified starch-containing material is converted in a single step process as part of a consolidated bioprocessing (CBP) system and wherein in particular the lactic acid is separated during and/or after the conversion.

29. The fermentation process according to claim 28, wherein the period of time is at least one of 10 hours to 300 hours, 40 hours to 200 hours, and 60 hours to 160 hours, wherein the initial temperature is in a range between at least one of 55° C. and 80° C., 70° C. and 78° C., and 72° C. and 74° C., wherein the initial pH is in a range between at least one of 5 and 9, 6 and 8, and 7 and 7.5.

30. The fermentation process according to claim 28, wherein the unmodified starch-containing material comprises unmodified starch-containing plant material.

31. The fermentation process according to claim 30, wherein the unmodified starch-containing material is corn (maize), wheat, pea, barley, cassava, sorghum, rye, potato, or any combination thereof.

32. The fermentation process according to claim 28, wherein the starch containing material is potato, parts of potato and/or potato-containing material.

33. The fermentation process according to claim 28, wherein the starch containing material is corn, parts of corn and/or corn-containing material.

34. The fermentation process according to claim 28, wherein the starch containing material is pea, parts of pea and/or pea-containing material.

35. The fermentation process according to claim 28, wherein the microorganism of the genus Caldicellulosiruptor is selected from the group consisting of Caldicellulosiruptor acetigenus, Caldicellulosiruptor bescii, Caldicellulosiruptor changbaiensis, Caldicellulosiruptor danielii, Caldicellulosiruptor sp. strain F32, Caldicellulosiruptor hydrothermalis, Caldicellulosiruptor kristjanssonii, Caldicellulosiruptor kronotskyensis, Caldicellulosiruptor lactoaceticus, Caldicellulosiruptor morganii, Caldicellulosiruptor naganoensis, Caldicellulosiruptor obsidiansis, Caldicellulosiruptor owensensis and Caldicellulosiruptor saccharolyticus like Caldicellulosiruptor sp. str. DIB 041C DSM 25771, Caldicellulosiruptor sp. str. DIB 004C DSM 25177, Caldicellulosiruptor sp. str. DIB 101C DSM 25178, Caldicellulosiruptor sp. str. DIB 103C DSM 25773, Caldicellulosiruptor sp. str. DIB 107C DSM 25775, Caldicellulosiruptor sp. str. DIB 087C DSM 25772, Caldicellulosiruptor sp. str. DIB 104C DSM 25774, Caldicellulosiruptor sp. BluCon006 DSM 33095, Caldicellulosiruptor sp. BluCon014 DSM 33096, Caldicellulosiruptor sp. BluCon016 DSM 33097 and Caldicellulosiruptor sp. BluConL60 DSM 33252.

36. The fermentation process according to claim 28, wherein the microorganism is Caldicellulosiruptor sp. BluConL60 (DSMZ Accession number 33252), microorganism derived therefrom, progenies or a mutant thereof, wherein the mutant thereof retaining the properties of BluConL60.

37. The fermentation process according to claim 28, wherein the lactic acid is L-lactic acid or D-lactic acid or the racemic mixture of D- and L-lactic acid.

38. A lactic acid production procedure, characterized in that it includes the following steps:
a) converting unmodified starch and/or unmodified starch-containing material to lactic acid in a single step process as part of a consolidated bioprocessing (CBP) system in a bioreactor by a microorganism of the genus Caldicellulosiruptor,
b) separation of lactic acid from the fermentation medium, and
c) purification of lactic acid.

39. The lactic acid production procedure according to claim 38, characterized in that it includes the following steps:
a) converting unmodified starch and/or unmodified starch-containing material to lactic acid in a single step process as part of a consolidated bioprocessing (CBP) system in a bioreactor by a microorganism of the genus Caldicellulosiruptor,
b) separation of lactic acid from the fermentation medium,
c) purification of lactic acid, and
d) where in step a) no amylolytic enzymes are added.

40. The lactic acid production procedure according to claim 38, characterized in that it includes the following steps:
a) converting unmodified starch and/or unmodified starch-containing material to lactic acid in a single step process as part of a consolidated bioprocessing (CBP) system in a bioreactor by a microorganism of the genus Caldicellulosiruptor,
b) separation of lactic acid from the fermentation medium
c) purification of lactic acid,
d) where in step a) no amylolytic enzymes are added, and
e) where in the unmodified starch and/or unmodified starch-containing material was not heat treated before the conversion in step a).

41. The lactic acid production procedure according to claim 38, characterized in that the simultaneous saccharification and fermentation occurs in a standard bioreactor with a stirrer, or a bioreactor with different supports, or a tower bioreactor, or a horizontal tubular bioreactor and other types of bioreactors.

42. The lactic acid production procedure according to claim 38, wherein the unmodified starch and/or unmodified starch-containing material is converted to lactic acid for a range between at least one of between 10 hours to 300 hours, 40 hours to 200 hours, and 60 hours to 160 hours,
wherein the unmodified starch and/or unmodified starch-containing material is converted to lactic acid at a temperature range between at least one of 55° C. and 80° C., 70° C. and 78° C., and 72° C. and 74° C., wherein the unmodified starch and/or unmodified starch-containing material is converted to lactic acid at a pH range between at least one of 5 and 9, 6 and 8, and 7 and 7.5.

43. The lactic acid production procedure according to claim 38, wherein the unmodified starch-containing material comprises unmodified starch-containing plant material.

44. The lactic acid production procedure according to claim 38, wherein the unmodified starch-containing material is corn (maize), wheat, pea, barley, cassava, sorghum, rye, potato, or any combination thereof.

45. The lactic acid production procedure according to claim 38, wherein the starch-containing material is potato, parts of potato and/or potato-containing material.

46. The lactic acid production procedure according to claim 38, wherein the starch containing material is corn, parts of corn and/or corn-containing material, or wherein the starch-containing material is pea, parts of pea and/or pea-containing material.

47. The lactic acid production procedure according to claim 38, wherein the microorganism of the genus Caldicellulosiruptor is selected from the group consisting of Caldicellulosiruptor acetigenus, Caldicellulosiruptor bescii, Caldicellulosiruptor changbaiensis, Caldicellulosiruptor danielii, Caldicellulosiruptor sp. strain F32, Caldicellulosiruptor hydrothermalis, Caldicellulosiruptor kristjanssonii, Caldicellulosiruptor kronotskyensis, Caldicellulosiruptor lactoaceticus, Caldicellulosiruptor morganii, Caldicellulosiruptor naganoensis, Caldicellulosiruptor obsidiansis, Caldicellulosiruptor owensensis and Caldicellulosiruptor saccharolyticus like Caldicellulosiruptor sp. str. DIB 041C DSM 25771, Caldicellulosiruptor sp. str. DIB 004C DSM 25177, Caldicellulosiruptor sp. str. DIB 101C DSM 25178, Caldicellulosiruptor sp. str. DIB 103C DSM 25773, Caldicellulosiruptor sp. str. DIB 107C DSM 25775, Caldicellulosiruptor sp. str. DIB 087C DSM 25772, Caldicellulosiruptor sp. str. DIB 104C DSM 25774, Caldicellulosiruptor sp. BluCon006 DSM 33095, Caldicellulosiruptor sp. BluCon014 DSM 33096, Caldicellulosiruptor sp. BluCon016 DSM 33097 and Caldicellulosiruptor sp. BluConL60 DSM 33252.
